(19) United States
(12) Patent Application Publication
(10) Pub. No.: US 2008/0003227 A1
(43) Pub. Date: Jan. 3, 2008

(54) Acetyl CoA Carboxylase Splice Variant and Uses Thereof

(75) Inventors: John Clapham, Molndal (SE); Brit Corneliussen, Molndal (SE); Stefan Hallen, Molndal (SE)

Correspondence Address: Pepper Hamilton LLP, 500 Grant Street, One Mellon Bank Center, 50th Floor, Pittsburgh, PA, 15219-2502, US

(73) Assignee: ASTRAZENECA AB, Sodertalje (SE)

(21) Appl. No.: 11/571,679

(22) PCT Filed: Jul. 6, 2005
(86) PCT No.: PCT/SE05/01121

§ 371 Date: Jan. 5, 2007

(30) Foreign Application Priority Data
Jul. 7, 2004 (GB) 0415196.5

(51) Int. Cl. A61K 39/395; A61K 31/711; A61P 3/04; A61P 3/10; C07K 14/435; C07K 16/40; C12N 1/19; C12N 1/21; C12N 15/52; C12N 15/63; C12N 15/85; C12N 5/10; C12N 9/00; C12P 21/00; C12Q 1/68; G01N 33/573

(52) U.S. Cl. 424/158.1; 435/252.3; 435/254.2; 435/320.1; 435/325; 435/348; 435/419; 435/6; 435/69.1; 435/7.8; 514/44; 530/350; 530/388.26; 536/23.2

(57) ABSTRACT

The present invention relates to an isolated nucleic acid molecule comprising a nucleotide sequence that encodes an acetyl CoA carboxylase 2 (ACC2) splice variant. It also relates the corresponding polypeptide encoded by said nucleic acid and to methods of identifying a compound potentially useful for treating diseases or disorders associated with impaired ability to oxidise fatty acids, which comprises assaying the compound for its ability to modulate the activity or amount of a ACC2 splice variant complex or a complex thereof.

### TECHNICAL FIELD

The present invention relates to an isolated nucleic acid molecule comprising a nucleotide sequence that encodes an acetyl CoA carboxylase 2 (ACC2) splice variant. It also relates the corresponding polypeptide encoded by said nucleic acid and to methods of identifying a compound potentially useful for treating diseases or disorders associated with impaired ability to oxidise fatty acids, which comprises assaying the compound for its ability to modulate the activity or amount of a ACC2 splice variant complex or a complex thereof.

### BACKGROUND OF THE INVENTION

A number of recent findings have pointed to alternative pre-mRNA splicing as a generator of protein diversity that in some cases rivals the immune system in the degree of diversity generated by a single gene. As many as 40-60% of human genes have been predicted to have alternative splice forms based on genome-wide analyses of alternative splicing. This process allows the optional inclusion or substitution of some exons within the constant framework provided by constitutive exons. The resultant protein isoforms commonly vary within specific functional domains while sharing common functions (Lopez A J. Annu Rev Genet, 32:279-305, 1998; Graveley. Trends Genet. 17:100-107, 2001). Alternative splicing may also lead to premature termination of open reading frames, or even use of the same mRNA sequence in two different reading frames (Quelle et al., Cell. 83:993-1000, 1995). Alternative splicing could therefore, have major functional consequences for the proteins and its global significance in generating protein diversity is due to its prevalence and the extreme combinatorial output of some genes.

The last 25 years have been witness to an alarming increase in the incidence and severity of obesity. This is occurring both in the developed nations and emerging Third World countries. For example, over half the U.S. population is affected by overweight. Excess body weight (BMI>27 in Western societies, >25 in Asia) is associated with increased risks for type 2 diabetes, hypertension, dyslipidaemia, cardiovascular disease, osteoarthritis and some cancers. Weight loss can have profound effects on many of these morbidities, for example progression to type 2 diabetes can almost be prevented. However, although significant and beneficial weight loss can be readily achieved, the greatest challenge is maintenance; weight regain is inevitable for the vast majority. Therefore, anti-obesity agents which specifically address this issue will satisfy a major unmet clinical need.

Skeletal muscle is responsible for a large proportion of whole body lipid oxidation and the primary fate of lipid delivered to muscle is for use as an oxidative fuel. Plasma non-esterified free fatty acids (NEFA) account for 80-90% of muscle fuel needs during fasting or mild exercise whilst circulating, inter- or intracellular triacylglyceride (TAG)-derived fatty acids account for most lipid oxidation when fuel demand is more sustained. Regulation of muscle lipid metabolism depends upon substrate availability and subsequent trafficking of free fatty acids within the cell. Delivery of free fatty acid substrate to muscle depends on the mobilisation and transport of free fatty acids originally esterified in the form of TAG. In obesity, the oversupply of free fatty acids drives metabolism towards TAG synthesis and storage in muscle. It is now well established that insulin resistance is tightly associated with excess intramyocellular TAG in skeletal muscle (Krssak et al., Diabetologia 42:113-6, 1999).

In the clinic, failure to maintain weight loss is strongly associated with impairment of fatty acid oxidation (Valtuena et al., Int J. Obes. Relat. Metab. Disord. 21:811-7, 1997a; Valtuena et al., Int. J. Obes. Relat. Metab. Disord. 21:267-732, 1997b) and represents a major hurdle in the pharmacotherapy of obesity. Conceptually, reduction of the malonyl CoA produced by acetyl CoA carboxylase 2 (ACC2) in oxidative tissues such as skeletal muscle would be expected to increase fatty acid oxidation rates and lower respiratory quotient (RQ). This may therefore improve the oxidative potential in obese patients after weight loss. In accordance, ACC2 has been put forward as a promising anti-obesity target since absence of ACC2 results in a lean phenotype in mice (Abu-Elheiga et al. Science 291:2613-2664, 2001) and its inhibition is a late step in leptin-induced increases in fatty acid oxidation via the lowering of malonyl CoA.

There are 2 known isoforms of acetyl CoA carboxylase (ACC), ACC1 (ACCα) which is cytosolic and predominantly expressed in liver and adipose tissue and ACC2 (ACCβ) which is believed to be associated with mitochondria and mainly expressed in skeletal muscle and heart. The acetyl CoA carboxylases catalyse the ATP-driven carboxylation of acetyl CoA to form malonyl CoA. Malonyl CoA produced by ACC1 is used in fatty acid synthesis while the malonyl CoA postulated to be formed by ACC2 locally on the mitochondrial surface regulates the palmitoyl CoA shuttle system (CPT-1). Malonyl CoA is a potent inhibitor of carnitine palmitoyl transferase 1 (CPT-1) and as a consequence it decreases the fatty acid flux into mitochondria. Thus, reduction of ACC2 activity would reduce local malonyl CoA levels and increase fatty acid β-oxidation concomitantly reducing triacylglycerol (TAG) synthesis (Munday. Biochem Soc Trans. 30:1059-64, 2001).

Thus, the acetyl CoA carboxylases (EC 6.4.1.2) belong to the enzyme family of carbon bond forming ligases. Using biotin as a cofactor, the ACCs carboxylate acetyl CoA in an ATP-driven multi-step reaction to form malonyl CoA:
ATP+HCO₃⁻+ACC-biotin→ADP+Pᵢ+ACC-biotin-CO₂ (1)
ACC-biotin-CO₂+acetyl CoA→ACC-biotin+malonyl CoA (2)

The human acetyl CoA carboxylase 2 is believed to be anchored by a hydrophobic N-terminus in the mitochondrial outer membrane (Abu-Elheiga et al., Proc Natl Acad Sci U S A. 97(4):1444-9, 2000). The enzyme has three functional domains on a 2458 amino acid large single polypeptide chain (˜280 kDa) working in concert to carry out the partial reactions shown above.

ACC1 is known to be short term regulated by polymerisation, de-polymerisation and phosphorylation. Citrate is considered the physiological allosteric activator of the ACCs and it has been demonstrated to induce a multi-step polymerisation of ACC1 protomers into a filamentous structure. Citrate binding causes an initial conformational change of the inactive ACC1 protomer that triggers a subsequent dimer formation (“dimers” constituted of 4 ACC1 peptides). This initial complex formation is believed to be the rate limiting step in the overall ACC polymerisation. ACC2 has also been demonstrated to be activated by citrate. The mechanism, however, is unclear as the attachment of the ACC2 monomer/protomer to the mitochondrial outer membrane is expected to restrict how the enzyme can polymerise. The newly discovered ACC2 splice variant, ACC2(1b), lacking the membrane binding sequence, is suggested to be the cytoplasmic partner in forming a citrate induced multimeric complex with ACC2.

It appears, however, as if the citrate activation of the carboxylase reaction precedes the appearance of both the dimeric and polymeric form of the enzyme. This finding implies that initiation of enzyme turnover is not dependent upon the enzyme complex formation and, thus, would not require the postulated ACC2-ACC2(1b) partnership in mitochondrial malonyl CoA formation. The function and importance of polymerisation is instead believed to be a structural protection of the ACC turnover from inactivating phosphorylation by the kinases, for example, AMPK and/or cAMP PK. Thus, the ACC2 protomer would, although initially activated by citrate, rapidly become accessible to the above mentioned kinases and turned off by the simultaneous phosphorylation unless being associated with ACC2(1b). Compounds preventing the ACC2-ACC2(1b) complex formation would therefore not prevent the activation per se but rather cause a downstream inhibition of the enzyme by the energy regulatory machinery of the cell. Thus, compounds interacting with ACC2 and/or ACC2(1b) in a way that blocks the citrate induced complex formation would be useful in slowing down malonyl CoA production on the mitochondrial surface and therefore also for increasing rate of fatty acid oxidation in mitochondria. There is precedent for heterodimerisation of ACC1 and ACC2 from immunoprecipitation studies. In one study (Dyck et al. Eur J Biochem 1999), the lower molecular weight product that co-precipitated with rat heart ACC2 was detected solely by using streptavidin-HRP, thus, identifying a biotinylated protein of ACC1 size, but not ACC1 directly. ACC2(1b) is expected to migrate at approximately the same rate as ACC1 when separated using SDS-PAGE. It should be noted, however, that the same investigators also show that ACC2 co-immunoprecipitates with ACC1 when a specific antibody against the latter is used. Further support for heterodimer formation comes from co-immunoprecipitation experiments of rat liver ACC1 and ACC2 (Winz et al, JBC 269(20):14438-14445, 1994) where a 1:1 complex composed of the two isoforms is reported. In addition, ACC1 and ACC2 were demonstrated to co-migrate when subjected to non-denaturing PAGE. The relatively low abundance of ACC1 in oxidative tissue like skeletal muscle and heart suggests that there is an alternative regulatory partner, e.g., ACC2(1b), in these organs.

The gene encoding ACC2 is located on chromosome 12 and the genomic sequence is disclosed in EMBL entry AC007637. Human ACC2 cDNA sequence is found in EMBL entry HSU89344. Alignment of the two sequences show that the human ACC2 gene consists of 52 coding exons. The alignment also identifies a number of errors in the published cDNA sequences of ACC2. Accordingly, the inventors have cloned and re-sequenced the ACC2 cDNA. The corrected sequence is depicted in SEQ ID NO:2. Related proteins. Genomic mouse sequence believed to represent the ACC2 gene demonstrates the highest homology (˜91%) with the human enzyme. Erroneous database sequence for rat ACC2 has 83% identity with the human ACC2. Human ACC1 and ACC2 are 76% identical (similarity ˜82%).

### SUMMARY OF THE INVENTION

The present invention arises from the discovery of a splice variant of ACC2, referred to herein as ACC2(1b). ACC2(1b) mRNA has been detected in human skeletal muscle, heart, fat tissue and liver. The splice variant lacks the membrane anchoring domain and is believed to be a regulatory partner of ACC2, forming the citrate induced polymer with the membrane anchored ACC2. In addition, ACC2(1b) is a potential mediator of the regulatory action of upstream/downstream kinases and phosphatases that constitute part of the malonyl CoA axis in fatty acid oxidation control. The object of the present invention is to provide a protein that has an important role in the understanding of metabolic diseases such as obesity, type 2 diabetes mellitus, dyslipidaemia, and other disorders associated with impaired ability to oxidise fatty acids. This object has been reached in that an isolated nucleic acid molecule is provided comprising a nucleotide sequence that encodes an acetyl CoA carboxylase 2 (ACC2) splice variant having the amino acid sequence according to SEQ ID NO: 1, or a variant having at least 85% sequence identity thereto, or a variant differing from the sequence disclosed in SEQ ID NO: 1, only by the substitition of synonymous codons, which variant lacks membrane binding ability. This splice variant can be used for the same screening, diagnostic, therapies etc. as wild-type ACC2.

According to further aspect of the invention, a plasmid comprising the above mentioned nucleic acid is provided. Further, there is provided a method for producing a polypeptide comprising the amino acid sequence of SEQ ID NO: 1, a sequence with at least 85% sequence identity thereto, or C-terminal truncated versions thereof, said polypeptide being incapable of membrane anchoring, the method comprising:

a) culturing a host cell containing an expression vector comprising a nucleic acid sequence which encodes a ACC2 splice variant polypeptide, or a polypeptide with at least 85% sequence identity thereto, or C-terminal truncated version thereof, which polypeptide is incapable of membrane anchoring, under conditions suitable for expression of the polypeptide; and

b) recovering the polypeptide from the host cell culture.

According to yet another aspect of the invention an isolated polypeptide is provided comprising the amino acid sequence depicted in SEQ ID No. 1, or a sequence possessing at least 85% similarity thereto, which polypeptide is incapable of membrane binding.

According to another aspect of the invention, a purified antibody is provided, capable of selectively binding to an ACC2 splice variant.

According to one aspect of the invention, there is provided the use of a compound able to modulate the activity or amount of ACC2 splice variant for the treatment of diseases or disorders associated with impaired ability to oxidise fatty acids.

According to another aspect of the invention, a method is provided of identifying a compound potentially useful for treating diseases or disorders associated with impaired ability to oxidise fatty acids, which comprises assaying the compound for its ability to modulate the activity or amount of an ACC2 splice variant protein.

According to another aspect of the invention, an isolated ACC2-ACC2(1b) protein complex is provided. Accordingly, a method is provided for identifying a compound potentially useful for treating diseases or disorders associated with impaired ability to oxidise fatty acids, which comprises assaying the compound for its ability to modulate the activity or amount of a complex comprising a ACC2 and an ACC2 splice variant.

Furthermore, the complex comprising wild-type ACC2 and ACC2(1b) splice variant constitutes the physiologically form of the enzyme that produces the malonyl CoA. Accordingly, this complex can now, for the first time, be prepared in vitro using recombinant technology. This complex can therefore be used in screens to identify compounds with potential therapeutic benefits in treating metabolic diseases such as obesity, type 2 diabetes mellitus, dyslipidaemia, and other disorders associated with impaired ability to oxidise fatty acids

Further, the use is provided of an inhibitory nucleic acid molecule selective against ACC2 splice variant nucleic acid or a selective antibody directed against ACC2 splice variant protein, in the manufacture of a medicament for treating diseases or disorders associated with impaired ability to oxidise fatty acids. In addition, there is provided the use of an inhibitory nucleic acid molecule selective against the ACC2 and ACC2(1b) splice variant complex, in the manufacture of a medicament for treating diseases or disorders associated with impaired ability to oxidise fatty acids.

### BRIEF DESCRIPTION OF THE FIGURES

FIG. 1 is a schematic representation the of human ACC2 gene structure including new exon1b. The gene spans approximately 130 kb and comprises 53 coding exons including two alternatively used start exons containing initiation ATGs, denoted as exon1a and exon 1b.

FIG. 2 shows the alignment of the amino acid sequences of human ACC2 and human ACC2 (1b).

FIG. 3 shows RNA expression levels of ACC2(1b) in human heart, liver, skeletal muscle, spleen and adipose tissue, measured using real-time PCR.

FIG. 4 shows the citrate stimulated activity of the expressed ACC2(1b) in transfected HEK293 cells.

FIGS. 5a to 5b show the results of SDS-PAGE and subsequent Western blotting analysis of cell lysates from ACC2, ACC2(1b) and mock-transfected HEK293 cells.

FIGS. 6a to 6f show immunostaining of human heart and skeletal muscle

### DETAILED DESCRIPTION OF THE INVENTION

Accordingly, the invention provides an isolated nucleic acid molecule comprising a nucleotide sequence that encodes an ACC2 splice variant having the amino acid sequence according to SEQ ID NO: 1, or a variant having at least 85% sequence identity thereto, or a variant differing from the sequence disclosed in SEQ ID NO: 1 only by the substitition of synonymous codons, which variant is incapable of membrane anchoring. The invention also provides a nucleic acid molecule comprising the complement of said sequences. The invention also provides expression vectors containing the claimed nucleic acid molecules and host cells transformed with said nucleic acids. The invention also provides expression systems for expressing both the wild-type (membrane anchoring) and splice variant forms of ACC2, so as to form the ACC2-ACC2(1b) complex. The invention also provides the use of this ACC2-ACC2(1b) complex in screens for compounds that modulate the activity of said complex. The invention also provides purified ACC2 splice variant polypeptide, particularly that having the amino acid sequence depicted in SEQ ID No: 1, those with at least 85% sequence identity thereto, or C- or N-terminal truncated versions thereof, which variants are incapable of membrane anchoring. The invention also provides assays for identifying compounds that modulate expression or activity of the ACC2 splice variant of the invention, which compounds may have therapeutic value, particularly in obesity, type 2 diabetes mellitus, dyslipidaemia, and other disorders associated with impaired ability to oxidise fatty acids.

According to a first aspect of the invention there is provided an isolated nucleic acid molecule comprising a nucleotide sequence that encodes ACC2 splice variant having the amino acid sequence according to SEQ ID NO: 1, or a variant having at least 85% sequence identity thereto, or a variant differing from SEQ ID NO: 1 only by the substitition of synonymous codons, wherein the variant lacks membrane anchoring ability. In one embodiment the ACC2 splice variant comprises the amino acid sequence disclosed in SEQ ID NO: 3.

According to another aspect of the invention there is provided an isolated nucleic acid molecule comprising a nucleotide sequence that encodes an ACC2 polypeptide having the N-terminal 16 amino acid sequence disclosed in SEQ ID NO: 3, or one with less than 4 amino acid substitutions within this 16 amino acid region, which variant is incapable of anchoring to a membrane. According to another aspect of the invention there is provided an isolated nucleic acid molecule comprising a nucleotide sequence that encodes an ACC2 polypeptide wherein the signal peptide/targeting peptide domain represented by positions 1 to 8 (SEQ ID NO: 4) is absent. As will be apparent to one skilled in the art, the degeneracy of the genetic code allows for numerous nucleotide substitutions in a given coding sequence which do not affect the amino acid sequence of the encoded protein. Thus the present invention also provides for isolated nucleic acids, which differ from any of the ACC2 splice variant encoding nucleotide sequences disclosed in the sequence listing only by substitution of such synonymous codons. The polymorph analysis of example 10 provides details of found differences between individuals.

In a particular embodiment of the invention the nucleic acid comprises a nucleotide sequence according to SEQ ID NO: 1, or a nucleotide with at least 85% sequence identity thereto, wherein said nucleic acid encodes an ACC2 splice variant polypeptide with the N-terminus depicted in SEQ ID NO:3. In another embodiment, the invention also encompasses a nucleotide sequence that encodes a variant of the polypeptide disclosed in SEQ ID NO: 3 wherein said variant has at least 81% sequence identity thereto. In one embodiment the nucleic acid comprises the nucleotide sequence depicted in SEQ ID NO:5.

In another aspect, the invention provides an isolated ACC2(1b) polypeptide. In one embodiment the polypeptide has the amino acid sequence according to SEQ ID NO: 1; or a sequence with at least 95% amino acid sequence identity thereto; or C-terminal truncated versions thereof. Particular embodiments are variants possessing the N-terminal sequence depicted in SEQ ID NO: 3.

In another aspect, the present invention provides ACC2 splice variant proteins in which conservative amino acid substitutions have been made for certain residues, to produce non-naturally occurring splice variants which retain ACC2 activity. Conservative substitutions are preferably sited in areas that have not been implicated in catalysis.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

In still another aspect, the invention provides a method for producing a polypeptide comprising the amino acid sequence of SEQ ID NO: 1, a sequence with at least 85% sequence identity thereto, or C-terminal truncated versions thereof, said polypeptide being incapable of membrane anchoring, the method comprising: a) culturing a host cell containing an expression vector comprising a nucleic acid sequence which encodes a ACC2 splice variant polypeptide, or a polypeptide with at least 85% sequence identity thereto, or C-terminal truncated version thereof, which polypeptide is incapable of membrane anchoring, under conditions suitable for expression of the polypeptide; and b) recovering the polypeptide from the host cell culture.

Such proteins can be recovered from the cells themselves, or from culture medium, if appropriate heterologous secretion signals (such as that from SUC2 or alpha-factor) are used to ensure secretion of the polypeptide into the culture medium.

In still another aspect, the invention provides a method for producing a polypeptide comprising the amino acid sequence of SEQ ID NO: 1, a sequence with at least 95% sequence identity thereto, or C-terminal truncated versions thereof, said polypeptide also comprising the sequence depicted in SEQ ID NO: 3. The method comprising: a) culturing a host cell containing an expression vector comprising a nucleic acid sequence which encodes a ACC2 splice variant polypeptide, or a polypeptide with at least 95% sequence identity thereto and comprising the N-terminal 16 amino acids depicted in SEQ ID NO:3, or C-terminal truncated version thereof, under conditions suitable for expression of the polypeptide; and b) recovering the polypeptide from the host cell culture.

Thus, in another aspect, the invention provides for cells and cell lines transformed with the nucleic acids of the present invention. The transformed cells may, for example, be mammalian, bacterial, yeast or insect cells.

According to a further aspect of the invention there is provided a method for preparing an ACC2-ACC2(1b) complex comprising culturing host cells capable of expressing wild-type and splice variant forms of ACC2 under conditions suitable for expression of the polypeptides, (b) allowing the polypeptides to form a complex; and, (c) recovering the complex.

According to another aspect of the invention there is provided isolated ACC2-ACC2(1b) protein complex. Said isolated protein complex may be present on, or associated with, membrane preparations or fractions.

According to another aspect of the invention there is provided use of recombinantly produced ACC2-ACC2(1b) complex in a screen for compounds with therapeutic potential.

According to another aspect of the invention there is provided use of isolated ACC2-ACC2(1b) complex in a screen for compounds with therapeutic potential.

In another aspect the invention provides a purified antibody, which selectively binds to the ACC2 splice variant, and methods for making antibodies which selectively bind with the ACC2 splice variant protein of the invention. By selectively binds we mean that it is substantially incapable of binding to native full-length ACC2 or any part thereof, or any other protein or polypeptide other that the ACC2 splice variant. The person skilled in the art is capable of identifying amino acid sequence differences between wild-type ACC2 and the various splice variant forms against which to design and prepare selective antibodies. The key difference lies in the N-terminal region. As used herein, in respect of antibody binding, the words ‘selective’ and ‘specific’ are used interchangeably.

In another aspect of the invention there is provided the use of a compound able to modulate the activity or amount of ACC2 splice variant for the treatment of diseases such as obesity, type 2 diabetes mellitus, dyslipidaemia, and other disorders associated with impaired ability to oxidise fatty acids.

Modulation of the amount of ACC2 splice variant of the invention by a compound may be brought about for example through altered gene expression level or message stability. Modulation of the activity of ACC2 splice variant by a compound may be brought about, for example, through compound binding to ACC2 splice variant protein, or the ACC2-ACC2(1b) complex. In one embodiment, modulation of ACC2 splice variant comprises a compound able to reduce the activity or amount of ACC2 splice variant. In another embodiment, modulation of ACC2 splice variant comprises a compound able to increase the activity or amount of ACC2 splice variant. In another embodiment, modulation of ACC2 activity is effected by a compound able to inhibit activity or amount of ACC2-ACC2(1b) complex, or complex formation per se. An example of a compound able to modulate the activity of ACC2 splice variant is an antibody. Antibodies can be prepared using any suitable method. For example, purified polypeptide may be utilized to prepare specific antibodies. The term “antibodies” is meant to include polyclonal antibodies, monoclonal antibodies, and the various types of antibody constructs such as for example F(ab′)₂, Fab and single chain Fv. Antibodies are defined to be specifically binding if they bind with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011; 4,902,614; 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., (Biotechnology. 7: 394, 1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the present invention there is provided use of a compound able to modulate the activity or amount of the ACC2 splice variant in the preparation of a medicament for the treatment of diseases such as obesity, type 2 diabetes mellitus, dyslipidaemia, and other disorders associated with impaired ability to oxidise fatty acids. Thus, the present invention also provides assays for identifying small molecules, or other compounds, which are capable of modulating the expression or activity of the ACC2 splice variant genes or proteins of the invention. Such assays may be performed in vitro using non-transformed cells, established cell lines or transformed cells of the invention, or in vivo using normal non-human animals or transgenic animals.

In particular, the assays may detect the presence of altered (increased or decreased) expression of the nucleic acids of the invention, increased or decreased levels of protein products encoded for by such nucleic acids, or increased or decreased activity of such a protein.

According to yet another aspect of the present invention there is provided a method of identifying a compound potentially useful for treatment of diseases such as obesity, type 2 diabetes mellitus, dyslipidaemia, and other disorders associated with impaired ability to oxidise fatty acids, which comprises assaying the compound for its ability to modulate the activity or amount of ACC2 splice variant. Preferably the assay is selected from:
i) measurement of ACC2 splice variant activity using a cell line which expresses ACC2 splice variant or using purified ACC2 splice variant protein;
ii) measurement of ACC2 activity using a cell line which expresses ACC2 splice variant and ACC2 wild-type or using purified ACC2-ACC2(1b) protein complex; and
iii) measurement of ACC2 splice variant transcription or translation in a cell line expressing ACC2 splice variant.

The method could be performed by using a complex of isolated ACC2 and an ACC2(1b) splice variant and measuring the activity of said complex with respect to malonyl CoA production. The measurements could be performed in a cell free assay using malachite green detection of produced inorganic phosphate formed by said complex or by measurement of the incorporation of ¹⁴CO₂ into ¹⁴C-malonyl CoA. The method could also be performed in a cell based assay by measurement of the activity of the ACC2 and ACC2(1b) splice variant complex using a cell line which expresses the ACC2 and ACC2(1b) splice variant complex. The measurement of the transcription and/or translation of the ACC2 and ACC2(1b) splice variant complex using a cell line which expresses the ACC2 and ACC2(1b) splice variant complex could be performed. In one embodiment, the disease or disorder is selected from the group consisting of obesity, type 2 diabetes mellitus, or dyslipidaemia.

The assay used to determine the effect of a compound to be tested on the transcription or translation of ACC2 splice variant can be based on:

i) measurement of the amount of ACC2 splice variant mRNA formed using e.g. Northern blot analysis or quantitative real time PCR,

ii) measurement of the amount of ACC2 splice variant protein formed using e.g. Western blot analysis, or immunochemical analysis such as ELISA, or

iii) measurement of ACC2 splice variant activity as described above, in cells expressing ACC2 splice variant.

The cells used in the assay can be cells naturally expressing an ACC2 splice variant or transfected cells expressing a recombinant ACC2 splice variant. Preferably the ACC2 splice variant is the human recombinant ACC2 splice variant.

The ACC2 splice variant, either alone or co-expressed with wild-type ACC2, may be expressed in a variety of hosts such as bacteria, plant cells, insect cells, fungal cells and human and animal cells. Eukaryotic recombinant host cells are especially preferred. Examples include yeast, mammalian cells including cell lines of human, bovine, porcine, monkey and rodent origin, and insect cells including Drosophila and silkworm derived cell lines. Cell lines derived from mammalian species which may be used and which are commercially available include, L cells L-M(TK-) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

The expression vector comprising a nucleic acid encoding ACC2 splice variant, either alone or co-expressed with wild-type ACC2, may be introduced into host cells to express the polypeptide(s) via any one of a number of techniques including calcium phosphate transformation, DEAE-dextran transformation, cationic lipid mediated lipofection, electroporation or infection.

The transfected host cells are propagated and cloned, for example by limiting dilution, and analysed to determine the expression level of recombinant ACC2 splice variant/wild-type ACC2. Identification of transformed host cells that express ACC2 splice variant, alone or with wild-type ACC2, may be achieved by several means including immunological reactivity with antibodies and/or the detection of biological activity using the assays described herein.

Transcriptional regulation of gene expression is mediated by specific DNA elements in the promoter that directs binding of transcription factors, which thereby mediate transcription of the gene. Eukaryotic transcription factors can be divided in two main groups i) basal transcription factors that interact with promoter sequences proximal to the start of transcription, thereby initiating transcription upon recruitment of RNA polymerase II and ii) transcription factors that bind to specific distal promoter elements, thereby modulating the transcription upon contact with the basal transcription machinery. A fundamental physiological process in the eukaryotic organism is that cells can communicate with their environment and respond to extracellular stimuli through signalling molecules, such as hormones and growth factors. The final event for such signalling is the binding of transcription factors to specific distal promoter elements leading to for example up-regulated or tissue specific gene expression. Because of their regulatory role, promoter elements are putative targets for screening of therapeutic agents.

Suitable host cells are cells known to express ACC2 splice variant or cells known to express transcription factors that can influence the transcription of ACC2 splice variant. Host cells transfected with DNA encoding specific transcription factors can preferably be used to study the interaction with defined transcription factors and the ACC2 splice variant promoter.

The assay used to determine the effect of a compound to be tested on the transcription of ACC2 splice variant can be based on measurement of the activity of the ACC2 splice variant promoter using a reporter gene system. The reporter gene system is an expression system comprising nucleic acid molecules constituting a ACC2 splice variant promoter, or fragments thereof, the expression system further comprising a reporter gene, the promoter and the reporter gene being positioned so that the expression of the reporter gene is regulated by the ACC2 splice variant promoter. The amount of reporter protein formed is used as an indication of the activity of the ACC2 splice variant promoter.

Suitable reporter genes that can be used for the construction of the reporter gene system are e.g. the firefly luciferase gene, the bacterial chloramphenicol acetyl transferase (CAT) gene, the β-galactosidase (β-GAL) gene, and the green fluorescent protein (GFP).

According to another aspect of the present invention there is provided a method of preparing a pharmaceutical composition which comprises:
i) identifying a compound as useful for treating diseases such as obesity, type 2 diabetes mellitus, dyslipidaemia, and other disorders associated with impaired ability to oxidise fatty acids according to a method as described herein; and
ii) mixing the compound or a pharmaceutically acceptable salt thereof with a pharmaceutically acceptable excipient or diluent.

The diagnostic or prognostic applications may exist based on determination of relative amounts of each splice variant form. Further method of treatment applications may be available by modulating the expression or amount of the various splice variant forms.

Thus, according to a further aspect of the invention, there is provided a method of determining an individual's susceptibility to develop diseases such as obesity, type 2 diabetes mellitus, dyslipidaemia, and other disorders associated with impaired ability to oxidise fatty acids, comprising measuring the relative amounts of wild-type and splice variant forms of ACC2 expressed by the individual and determining the individual's susceptibility to develop diseases such as obesity, type 2 diabetes mellitus, dyslipidaemia, and other disorders associated with impaired ability to oxidise fatty acids based on relative amounts present.

According to a further aspect of the invention, there is provided a method of diagnosing the severity of diseases such as obesity, type 2 diabetes mellitus, dyslipidaemia, and other disorders associated with impaired ability to oxidise fatty acids in a patient, comprising measuring the relative amounts of wild-type and splice variant forms of ACC2 expressed by the individual and determining the severity of the disease based the on relative amounts present.

In one embodiment, the amount of the ACC2 forms is measured from a muscle biopsy sample. In a further embodiment the diagnostic method can be used to gauge the type of therapeutic treatment. In a further embodiment, the method can be used to assess the effectiveness of therapeutic treatment of an individual by monitoring the relative amount of full-length and splice variant forms of ACC2 expressed by the individual during therapeutic treatment. For example, by monitoring before, during and after treatment.

In another aspect, the present invention also provides methods of diagnosing individuals with disorders associated with impaired ability to oxidise fatty acids comprising assaying individuals for the presence of, or relative amounts of, the ACC2 splice variant of the present invention. In a particular embodiment, the disorder is obesity. Suitable assays include nucleic acid based assays (employing the nucleic acids of the present invention or those capable of specifically identifying the nucleic acids of the present invention) and protein based assays (employing the antibodies or polypeptides of the present invention).

In yet another aspect of the present invention there is provided a diagnostic method comprising the analysis of the sequence of the ACC2 splice variant gene, or a selective part thereof, in a DNA sample obtained from a patient, for the determination of susceptibility to develop diseases such as obesity, type 2 diabetes mellitus, dyslipidaemia, and other disorders associated with impaired ability to oxidise fatty acids.

Knowledge of the gene according to the invention also provides the ability to regulate its expression in vivo by for example the use of antisense DNA or RNA. One therapeutic means of inhibiting or dampening the expression levels of a particular gene or gene transcript (for example ACC2 splice variant identified herein) is to use antisense therapy. Antisense therapy utilises antisense nucleic acid molecules that are synthetic segments of DNA or RNA (“oligonucleotides”), designed to mirror specific mRNA sequences and block protein production. Once formed, the mRNA binds to a ribosome, the cell's protein production “factory” which effectively reads the RNA sequence and manufactures the specific protein molecule dictated by the gene. If an antisense molecule is delivered to the cell (for example as native oligonucleotide or via a suitable antisense expression vector), it binds to the messenger RNA because its sequence is designed to be a complement of the target sequence of bases. Once the two strands bind, the mRNA can no longer dictate the manufacture of the encoded protein by the ribosome and is rapidly broken down by the cell's enzymes, thereby freeing the antisense oligonucleotide to seek and disable another identical messenger strand of mRNA.

With knowledge of the ACC2 splice variant gene and mRNA sequence taught herein, the person skilled in the art is able to design suitable antisense nucleic acid therapeutic molecules and administer them as required.

Antisense oligonucleotide molecules with therapeutic potential can be determined experimentally using well-established techniques. To enable methods of down-regulating expression of the ACC2 splice variant gene of the present invention in mammalian cells, an example antisense expression construct can be readily constructed for instance using the pREP10 vector (Invitrogen Corporation). Transcripts are expected to inhibit translation of the gene in cells transfected with this type of construct. Antisense transcripts are effective for inhibiting translation of the native gene transcript, and capable of inducing the effects (e.g., regulation of tissue physiology) herein described. Oligonucleotides, which are complementary to and hybridisable with any portion of ACC2 splice variant gene mRNA are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from the ACC2 splice variant gene sequence are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

As noted above, antisense nucleic acid molecules may also be provided as RNAs, as some stable forms of RNA are now known in the art with a long half-life that may be administered directly, without the use of a vector. In addition, DNA constructs may be delivered to cells by liposomes, receptor mediated transfection and other methods known to the art.

The antisense DNA or RNA for co-operation with the target gene can be produced using conventional means, by standard molecular biology and/or by chemical synthesis as described above. If desired, the antisense DNA or antisense RNA may be chemically modified so as to prevent degradation in vivo or to facilitate passage through a cell membrane and/or a substance capable of inactivating mRNA, for example ribozyme, may be linked thereto and the invention extends to such constructs.

The antisense DNA or antisense RNA may be of use in the treatment of diseases or disorders in humans in which the over- or under-regulated production of the ACC2 splice variant gene product has been implicated.

Alternatively, ribozyme molecules may be designed to cleave and destroy the ACC2 splice variant mRNA in vivo. Ribozymes are RNA molecules that possess highly specific endoribonuclease activity. Hammerhead ribozymes comprise a hybridising region, which is complementary in nucleotide sequence to at least part of the target RNA, and a catalytic region, which is adapted to recognise and cleave the target RNA. The hybridising region preferably contains at least 9 nucleotides. The design, construction and use of such ribozymes is well known in the art and is more fully described in Haselhoff and Gerlach, (Nature. 334:585-591, 1988). In another alternative oligonucleotides designed to hybridise to the 5′-region of the ACC2 splice variant gene so as to form triple helix structures may be used to block or reduce transcription of the ACC2 splice variant gene. In another alternative, RNA interference (RNAi) oligonucleotides or short (18-25 bp) RNAi ACC2 splice variant sequences cloned into plasmid vectors are designed to introduce double stranded RNA into mammalian cells to inhibit and/or result in the degradation of ACC2 splice variant messenger RNA. ACC2 splice variant RNAi molecules may begin adenine/adenine (AA) or at least (any base-A, U, C or G) A . . . and may comprise of 18 or 19 or 20 or 21 or 22 or 23, or 24 or 25 base pair double stranded RNA molecules with the preferred length being 21 base pairs and be specific to individual ACC2 splice variant sequences with 2 nucleotide 3′ overhangs or hairpin forming 45-50mer RNA molecules. The design, construction and use of such small inhibitory RNA molecules is well known in the art and is more fully described in the following: Elbashir et al., (Nature. 411(6836):494-498, 2001); Elbashir et al., (Genes & Dev. 15:188-200, 2001); Harborth, J. et al. (J. Cell Science 114:4557-4565, 2001); Masters et al. (Proc. Natl. Acad. Sci. USA 98:8012-8017, 2001); and, Tuschl et al., (Genes & Dev. 13:3191-3197, 1999).

According to a further aspect of the invention there is provided a method of treating a human in need of treatment with a small molecule drug acting on the ACC2 splice variant protein or the ACC2-ACC2(1b) protein complex, or an inhibitory nucleic acid molecule acting against the ACC2 splice variant mRNA, in which the method comprises:

i) measuring the level of the ACC2 splice variant mRNA in suitable sample obtained from the human;

ii) determining the status of the human by reference to normal levels of the ACC2 splice variant mRNA; and,

iii) administering an effective amount of the drug or inhibitory nucleic acid molecule.

According to a further aspect of the invention there is provided a method of treating a human in need of treatment with a small molecule drug acting on the ACC2 splice variant protein or the ACC2-ACC2(1b) protein complex, or an inhibitory nucleic acid molecule acting against the ACC2 splice variant mRNA, in which the method comprises:

i) measuring the level of the ACC2 splice variant protein or ACC2-ACC2(1b) protein complex in a sample obtained from the human and,

ii) determining the status of the human by reference to normal levels of said proteins; and,

iii) administering an effective amount of the drug or inhibitory nucleic acid molecule acting against the ACC2 splice variant mRNA.

A method of treatment of a patient suffering from obesity, type 2 diabetes mellitus, dyslipidaemia, or other disorders associated with impaired ability to oxidise fatty acids, comprising administration to the patient of a compound or nucleic acid molecule capable of reducing the transcription or expression of ACC2 splice variant.

A method of treatment of a patient suffering from obesity, type 2 diabetes mellitus, dyslipidaemia, or other disorders associated with impaired ability to oxidise fatty acids, comprising administration to the patient an inhibitory nucleic acid molecule targeted against the MRNA of ACC2 splice variant.

Use of an inhibitory nucleic acid molecule against ACC2 splice variant nucleic acid or an antibody directed against ACC2 splice variant protein, in the manufacture of a medicament for treating diseases such as obesity, type 2 diabetes mellitus, dyslipidaemia, and other disorders associated with impaired ability to oxidise fatty acids.

In each of the above aspects of the invention, the “inhibitory nucleic acid molecule” is selected from the group consisting of: an antisense, ribozyme, triple helix aptemer and RNAi molecule.

Unless defined otherwise, all technical and scientific terms used herein have the same meanings as commonly understood by one of ordinary skill in the art to which this invention belongs. All publications mentioned herein are incorporated herein by reference.

### EXPERIMENTAL SECTION

The invention will now be illustrated with reference to the following non-limiting Examples.

AMPLITAQ™, available from Perkin-Elmer Cetus, is used as the source of thermostable DNA polymerase.

The practice of the present invention will employ, unless otherwise indicated, conventional methods of virology, immunology, microbiology, molecular biology and recombinant DNA techniques within the skill of the art. Such techniques are explained fully in the literature. See, e.g., Sambrook et al., eds., Molecular Cloning: A Laboratory Manual (3^{rd} ed.) Cold Spring Harbor Laboratory Press, Cold Spring Harbor, N.Y. (2001); Ausubel et al., eds., Current Protocols in Molecular Biology, John Wiley & Sons, New York, N.Y. (2002); Glover & Hames, eds., DNA Cloning 3: A Practical Approach, Vols. I, II, & III, IRL Press, Oxford (1995); Colowick & Kaplan, eds., Methods in Enzymology, Academic Press; Weir et al., eds., Handbook of Experimental Immunology, 5^{th} ed., Blackwell Scientific Publications, Ltd., Edinburgh, (1997); Fields, Knipe, & Howley, eds., Fields Virology (3^{rd} ed.) Vols. I & II, Lippincott Williams & Wilkins Pubs. (1996); Flint, et al., eds., Principles of Virology: Molecular Biology, Pathogenesis, and Control, ASM Press, (1999); Coligan et al., eds., Current Protocols in Immunology, John Wiley & Sons, New York, N.Y. (2002).

It is understood that this invention is not limited to the particular methodology, protocols, cell lines, vectors, and reagents described, as these may vary.

### Example 1

In silico prediction of a novel splice variant of human acetyl-coa carboxylase 2, ACC2(1b).

A sequence for a novel splice variant of human ACC2 was identified using a combination of novel sequence information and in silico prediction methods. The EMBL entry for human ACC2, HSU89344, was blasted against EMBL entries containing human genomic DNA (Blast2 NCBI). A human BAC clone from chromosome 12, RCPI11-443D10, was found to contain the gene encoding human ACC2. The genomic sequence of the clone is found in EMBL entry AC007637.

The gene structure of human ACC2 was analysed using alignments between HSU89344 and AC007637. Exons were predicted using alignments and exact exon borders were predicted using intron splice consensus sites. The alignments showed that the human ACC2 gene consists of 52 coding exons. Multiple mismatches were found between the sequence of HSU89344 and genomic sequences of AC007637. A corrected sequence for human ACC2 was produced by pasting the sequences of the 52 coding exons together in order to obtain a predicted coding sequence of ACC2. In one case, a part of the sequence of HSU89344 was predicted to consist of intron sequences flanked by atypical splice signals, and was therefore deleted from the predicted corrected coding sequence for human ACC2.

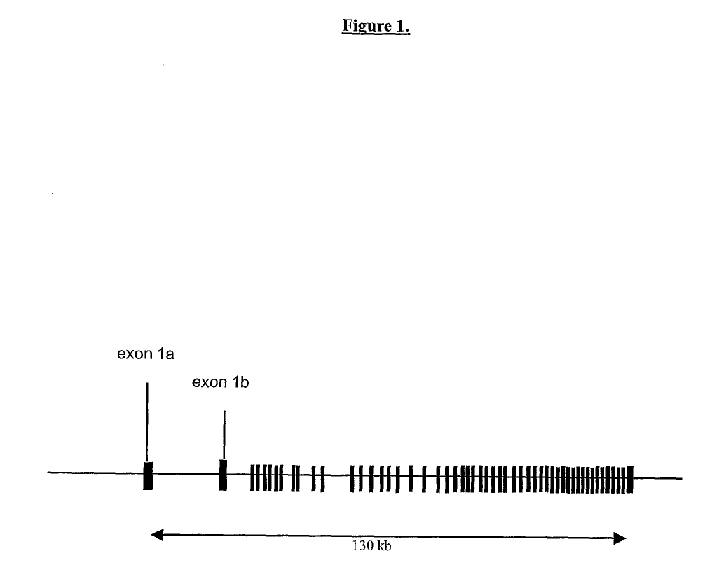

A Blast2 (NCBI) analysis using the predicted human ACC2 sequence towards the EST part of EMBL showed that there were two murine EST sequences with a novel 5′ end spliced to the sequence of exon 2 of the gene (murine ESTs BB854145 and BB866065). An alignment between the novel 5′ end of these two ESTs and human genomic DNA showed that the sequence is conserved in humans. Also, this conserved sequence was found to contain an alternative start ATG and to be followed by a 5′ consensus splice site both in mouse and human genomic DNA. Therefore, this sequence appeared to encode an alternatively used unique start exon of ACC2. This exon is referred to as exon 1b (as opposed to exon 1a containing the start ATG used in HSU89344) and a schematic gene structure including the new exon is shown in FIG. 1.

The coding part of the novel splice variant of human ACC2(1b) was constructed by splicing the coding sequence of exon 1b to the sequences of predicted exons 2-52 (predicted nucleotide sequence and amino acid sequence for the novel splice form is shown in SEQ ID NO: 5 and 1, respectively).

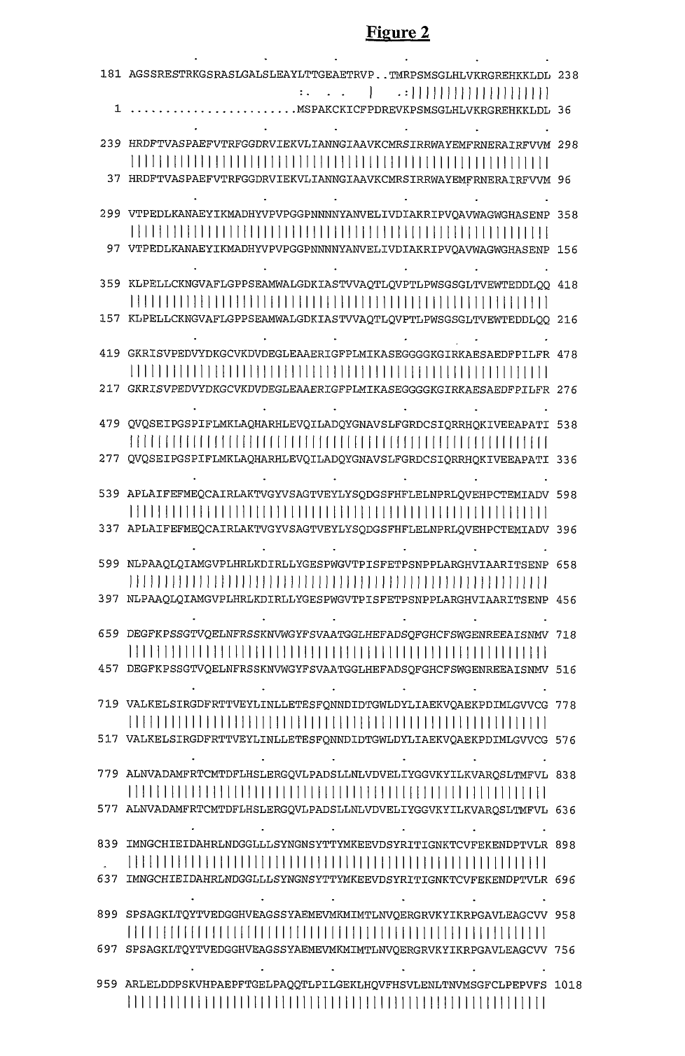

In FIG. 2, the alignment is shown of the amino acid sequences of human ACC2 and human ACC2 (1b). The alignment was performed using GCG Gap (Wisconsin) with the following parameters:

| | | | |
|---|---|---|---|
| Gap Weight: | 8 | Average Match: | 2.912 |
| Length Weight: | 2 | Average Mismatch: | −2.003 |
| Quality: | 11665 | Length: | 2461 |
| Ratio: | 5.171 | Gaps: | 1 |
| Percent Similarity: | 99.512 | Percent Identity: | 99.423 |

### Example 2

### Cloning of Human ACC2

In order to develop means for the production of recombinant ACC2, cDNA coding for human ACC2 was cloned by RT-PCR from human skeletal muscle mRNA. Total of three fragments covering the 7.5 kb sequence of ACC2 was PCR amplified and multiple clones were sequenced.

The first fragment, from 1 to 3925 was PCR amplified from a human heart cDNA library using the proof-reading polymerase pfu (Stratagene). The primers used for PCR were 5′-ATGGTCTTGCTTCTTTGTCTATC-3′ (SEQ ID NO: 6) as forward primer and 5′-GGCTGTTTAACACATAGGCGA-3′ (SEQ ID NO: 7) as reverse primer respectively. The product was cloned into the “TA” cloning vector pCR2.1, transformed into E. coli XL-10 Gold cells (Stratagene) and was fully double stranded sequenced.

The second and the third fragments, from 3250 to 5356 bp and 5335 to 7302 bp respectively, were PCR amplified from human skeleton muscle cDNA using Taq-plus Precision (Stratagene). For the second PCR fragment, 5′-CAGAGCATGGTGCAGTTGGT-3′ (SEQ ID NO: 8) was used as forward primer and 5′-CCATGTCTTCCAGGAGAGGTCC-3′ (SEQ ID NO: 9) as reverse primer. For the third PCR fragment, 5′-TCGTCATCGGCAATGACATA-3′ (SEQ ID NO: 10) was used as forward primer and 5′-GGTCCACCTCCGGCCC-3′ (SEQ ID NO: 11) as reverse primer. The PCR products were cloned into pCR2.1-TOPO vector and transformed into E. coli Top 10 cells (Invitrogen) and double strand sequenced.

The results showed that the nucleotide sequences of all clones differed significantly with the published cDNA sequence (accession number NM_{—}001993), but matched reasonably well with genomic sequence from human genome project as well as ESTs found in the public database.

The full-length ACC2 cDNA was pasted together by ligation of the three fragments digested out from the pCR2.1-TOPO vector. These fragments include 1 to 3288 bp, 3288 to 5323 bp and 5323 to 7302 bp and BamH1 site was used to link the two internal sites together. The full-length cDNA was ligated into mammalian expression vector pcDNA3.1 (+).

Transient transfection of pcDNA-ACC2 into COS-7 cells and HEK293 cells resulted in high levels of ACC2 expression as detected by antibodies against human ACC2.

In silico predictions of N-terminal sorting signals in the human ACC2 N-terminal region were performed using the iPSORT predictive software (Bannai et al., Bioinformatics 18:2, 298-305, 2002). Human ACC2 (SEQ ID NO:2) and in silico N-terminal truncations of human ACC2 were analysed and the predictions generated by iPSORT. The full length human ACC2 is predicted to encompass a signal peptide. Truncation of the sequence: MVLLL changes the iPSORT prediction from “signal peptide” to “mitochondrial transit peptide”. Further N-terminal truncation, excluding the sequence: MVLLLCL, results in the loss of targeting signature at the human ACC2 N-terminus.

### Example 3

### TaqMan Analysis of Human Tissues Verify the Presence of ACC2(1b) Transcript

Oligonucleotide primers and probes for hACC2(1b) were designed using the Primer Express 1.5 software (Applied Biosystems). Forward primer 5′-AGTCCTGCCAAGTGCAAGATCT-3′ (SEQ ID NO: 12), reverse primer 5′-TCTGTGCAGGTCCAGCTTCTT-3′ (SEQ ID NO: 13), and FAM-labeled probe 5′-TGATCGCGAAGTAAAGCCGAGCATGT-3′ (SEQ ID NO: 14) were design to cover exon 1B and exon 2. Human acidic ribosomal phosphoprotein (h36B4) primers and VIC-labelled probe were used as endogenous control.

The first strand cDNA was synthesized using Superscript III (Invitrogen) and oligoDt primers from 100 ng Poly A+ RNA (adipose tissue) and 1 μg total RNA (heart muscle, skeletal muscle and liver). To determine the expression of ACC2(1b) in human heart muscle (Ambion), skeletal muscle (Ambion), liver (BD Biosciences) and adipose tissue (BD Biosciences) real-time PCR (ABI prism 7500 detection system, Applied Biosystems) was used. PCR was performed by using Taqman Universal PCR Mastermix (Applied Biosystems) to which primers and probes were added and the expected size of the real-time PCR products was verified on a 3% agarose gel. The 7500 Real Time PCR System Sequence Detection Software (Applied Biosystems) was used for the analysis. Results in FIG. 3 show ACC2(1b) transcript levels in adipose tissue>skeletal muscle>heart muscle=liver with little or no variation between the samples (n=3).

### Example 4

### Cloning of Human ACC2(1b)

In order to make the ACC2(1b) clone we used the fact that only exon 1 differs between ACC2 and the splice variant ACC2(1b). A vector containing cDNA encoding human ACC2 with corrected sequence (SEQ ID NO: 2) was used as starting material. Cleavage sites for two restriction enzymes, Nhe1 and Hind3, were used to cut out the sequence for the ACC2 N-terminus and to substitute it with a fragment encoding the unique ACC2(1b) N-terminus. The Nhe1 recognition sequence was present in the multicloning region of the vector just upstream of the ACC2 start codon while the Hind3 site was present in the downstream natural sequence shared between ACC2 and ACC2(1b). The cDNA encoding the unique ACC2(1b) N-terminus and also including the succeeding shared ACC2/ACC2(1b) sequence was amplified from human heart cDNA using Taqplus® precision (Stratagene). The forward PCR primer containing the Nhe1 site was 5′-ATAAGCTAGCGCCACCATGAGTCCTGCCAAGTGCA-3′ (SEQ ID NO: 15) and the reverse primer downstream of the natural Hind3 site was 5′-TCCTCCGCACTCTCAGCCTTCCGGATT-3′ (SEQ ID NO: 16).

Both the vector encompassing the ACC2 cDNA and the PCR product, using the above mentioned PCR primers, were digested with the restriction enzymes, Nhe1 and Hind3, the cleaved products cleaned and the insert (PCR product) was then ligated into the vector. The resulting ligation product was transformed into E. coli TOP-10 cells and the insertion of the ACC2(1b) sequence into the vector was verified by dideoxy sequencing.

### Example 5

### Functional Expression of ACC2(1b) in HEK293 Cells

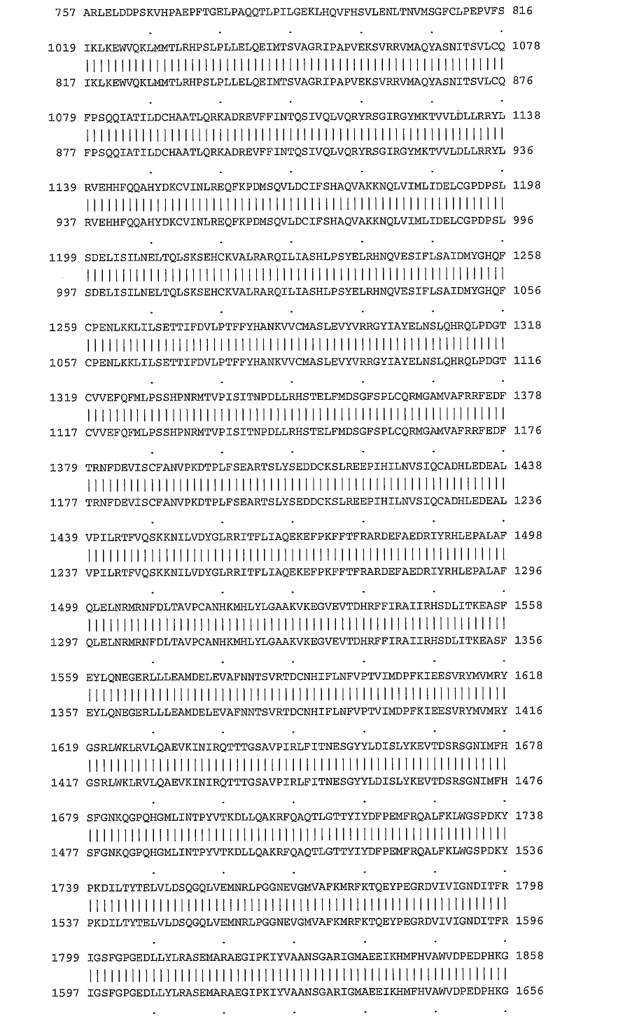

A vector, pcDNA3.1(+), encompassing the cDNA described in Examples 1 and 3, was used for transient transfection of HEK (human embryonic kidney) 293 cells. Cells were transfected with 5 μg of the abovementioned plasmid/10 cm Petri dish using a lipophilic transfection reagent. Cell monolayers were ˜60% confluent at the time of transfection (day 1) and fully confluent when harvested (day 4). Cell lysates from these transfected HEK293 cells were monitored for acetyl CoA carboxylase activity using a ¹⁴CO₂-fixation assay. The data presented in FIG. 4 shows the citrate stimulated activity of the expressed ACC2(1b). SDS-PAGE analysis of the ACC2(1b) cell lysate along with lysate from cells transfected with ACC2 showed the expected difference in molecular weight between the two proteins, the former migrating slightly faster than the full length ACC2 (data not shown). Thus, the ACC2 splice variant, ACC2(1b), demonstrates citrate stimulated catalytic activity as well as the expected higher migration rate during gel electrophoresis. Comparative Western analysis using antibodies specifically recognising N-terminal ACC2 sequence (epitope=a.a. 45-64) as well as an antibody raised against a mutual ACC2/ACC2(1b) sequence (epitope=a.a. 1335-1354) was performed. The former antibody recognized only the ACC2 peptide while the latter identified both the ACC2 and ACC2(1b) peptides, thus, confirming the N-terminal sequence differences between the two proteins.

### Example 6

### ACC2(1b) Antibody

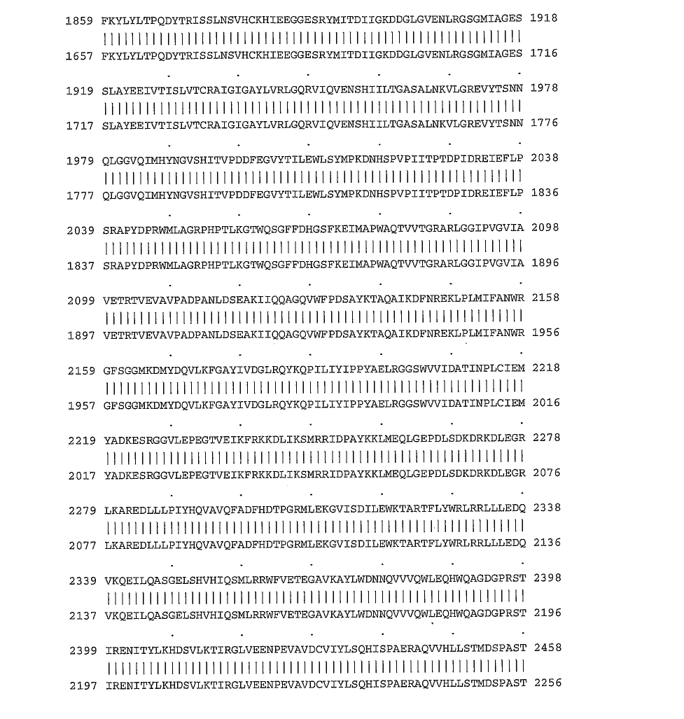

A synthetic peptide, MSPAKCKICFPDREVK (SEQ ID NO: 3), identical to the unique human ACC2(1b) N-terminus, was used in immunisation of rabbits. The same peptide was conjugated to a resin and utilized in affinity purification of produced serum, thus yielding an IgG fraction with specific recognition of ACC2(1b). Cell lysates from ACC2, ACC2(1b) and mock-transfected HEK293 cells were subjected to SDS-PAGE and the proteins transferred to PVDF for Western blotting using the produced antibody diluted 1:5000 (secondary antibody=goat anti rabbit HRP conjugated diluted 1:25000). Detection using ECL Plus (chemiluminescence) and subsequent exposure to film identified a specific band corresponding to ACC2(1b) without any recognition in neither the mock transfected cell lysate nor the ACC2 transfected cell lysate, although the ACC2 peptide is present as confirmed by Coomassie staining (FIG. 5).

### Example 7

### Immunostaining of Human Heart and Skeletal Muscle

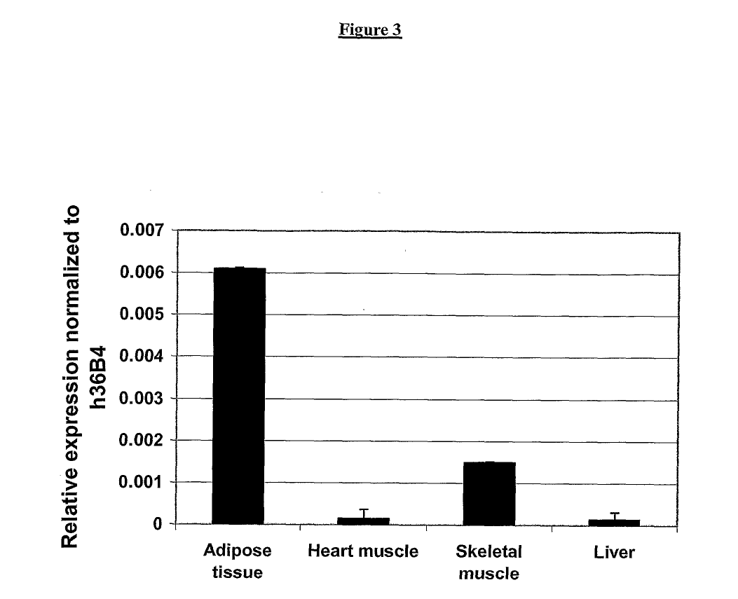

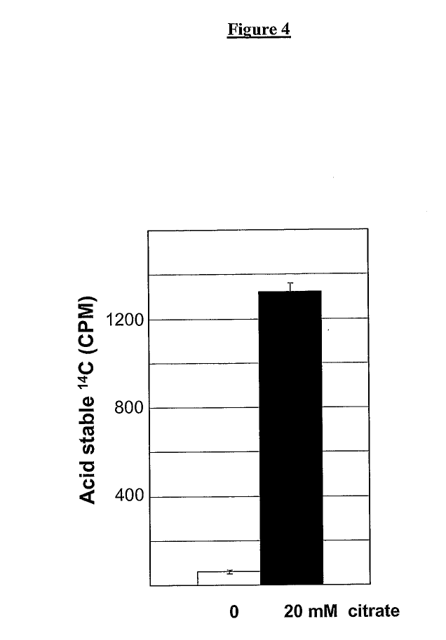

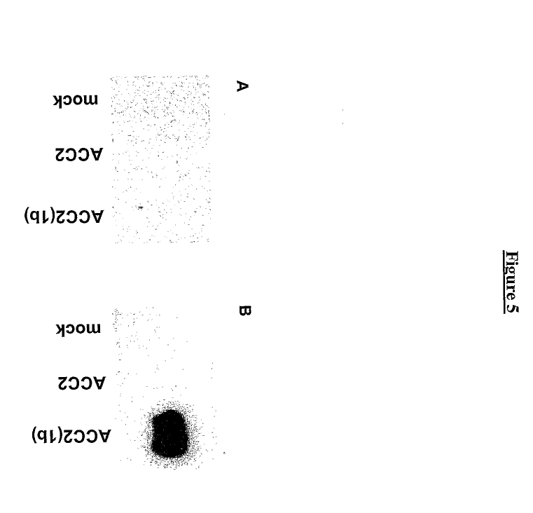

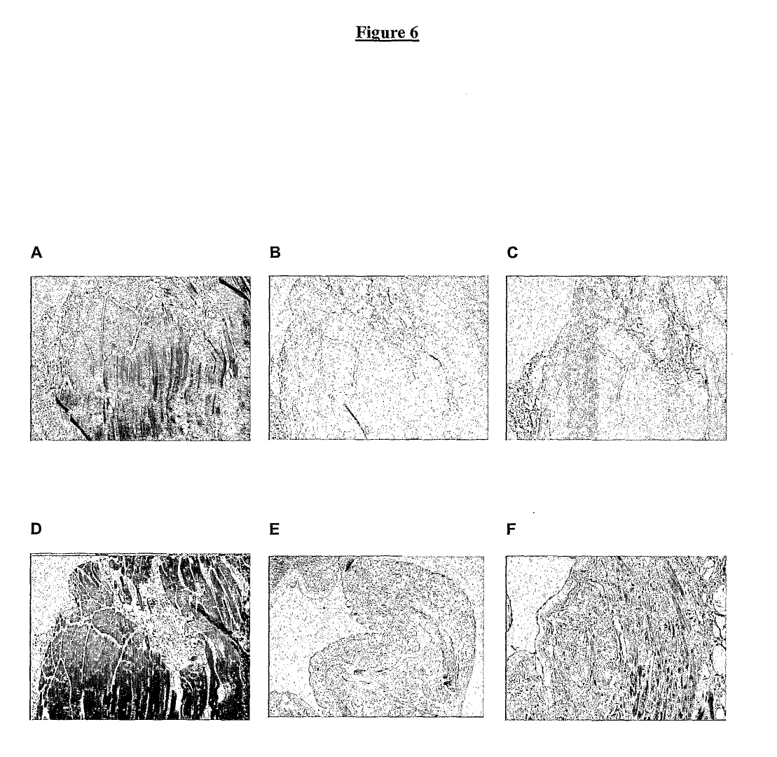

FIG. 6 shows human skeletal muscle stained with the ACC2(1b) antibody (1:100 dilution) described in example 6 in combination with a goat anti rabbit antibody from Ventana (HRP-conjugate, 1:500 dilution). Staining was made in the absence (FIG. 6A) or presence (FIG. 6B) of 10-fold excess synthetic peptide MSPAKCKICFPDREVK (SEQ ID NO:3) or without primary antibody (FIG. 6C). Comparison of FIGS. 6A and 6B show that pre-absorption with peptide blocks the staining and thereby demonstrates the specific interaction between the ACC2(1b) antibody and the endogenous enzyme. Furthermore, FIG. 6C demonstrates that the staining is mediated solely by the primary antibody as no colour develops in the presence of the secondary antibody alone. However, immunostaining of human skeletal muscle using an antibody (ACC2-4, diluted 1:100) recognizing both the full length ACC2 and the splice variant, ACC2(1b), was not blocked by pre-absorption with the peptide MSPAKCKICFPDREVK (SEQ ID NO:3) (FIG. 6D), further supporting the specificity in the interaction between the ACC2(1b) antibody and the endogenous epitope, ACC2(1b). FIGS. 6E and F represents ACC2(1b)-Ab staining of human heart muscle (atrial) using a 1:100 dilution. Thus, the ACC2(1b) splice variant protein has been shown to exist in nature since ACC2(1b) can be detected by immunostaining of native human oxidative tissues such as skeletal muscle and heart.

### Example 8

### ACC2-ACC2(1b) Complex Formation

Human ACC2 is expressed with a His-tag fused on to its C-terminus (hACC2-6xHis). A cell lysate containing the expressed fusion protein is allowed to equilibrate with and bind to a nickel resin column in the absence of citrate. A cell lysate encompassing the recombinant human ACC2(1b) is passed through the column in a medium containing an appropriate amount of citrate to induce the ACC2 conformational transfer and the subsequent complex formation between the ACC2 bound to the nickel resin and the added, soluble ACC2(1b). After washing off any excess of ACC2(1b) using the equilibration buffer, an eluting buffer containing an appropriate concentration imidazole is added and the ACC2-ACC2(1b) product is collected and used for testing compounds. Cell lines transformed to express physiological relevant levels of both ACC2 and ACC2(1b) can be established.

### Example 9

### Screening of Compounds Inhibiting Malonyl CoA Production by the ACC2-ACC2(1b) Complex

Inhibition of the ACC2-ACC2(1b) complex in a cell free assay can be monitored using both malachite green detection of produced inorganic phosphate formed during catalysis and by direct measurement of the incorporation of ¹⁴CO₂ into acid stable ¹⁴C-malonyl CoA. The effect of compounds in whole cells expressing the recombinant human ACC2-ACC2(1b) complex is determined from monitoring malonyl CoA concentrations in cell lysates from treated versus control cells. Malonyl CoA concentration is detected using an HPLC based protocol adapted for medium throughput screening.

### Example 10

### Investigation of the Polymorphism of the ACC2 Gene

Liver RNAs were obtained from 14 individuals from the USA of European origin and ACC2 cDNA was prepared from each sample using a primer specific to ACC2 (7336-7355). To obtain the most common sequence of ACC2, 11 overlapping PCR products were made from the individual cDNAs. Forward primers were tagged with M13F sequence and reverse primers with M13R sequence. Exons 1, 36, 41 and 51 were also amplified from genomic DNA from 5 unrelated Europeans and from a pool of DNA from 60 unrelated Europeans. Individual PCR products were sequenced in both directions by dye-terminator sequencing using M13F and M13R primers. Sequence traces were analysed for polymorphisms after assembly and quality calling with polyphred/phrap/consed package. Seqman was used to align the consensus sequence with the sequence disclosed in SEQ. ID 2. In order to identify the most common haplotype of ACC2 exons 11, 42 and 45 were also sequenced from genomic DNA in 28 lymphoblastoid cell lines from unrelated Europeans.

TABLE 1
Variants at specified positions on SEQ ID No. 2.

| | | Amino Acid | Allele | | Database |
|---|---|---|---|---|---|
| Position | SNP | Change | Frequency | | reference |
| 525 | T-C | Silent | 42% | | rs2878960 |
| 816 | C-T | Silent | 32% | | Incyte 00018514 |
| 1654 | A-G | I552V | 4% | (1/24) | |
| 1791 | C-T | Silent | 21.5% | | Incyte 00112383 |
| 1951 | G-A | A651T | 24% | (20/84) | rs2300455 |
| 2184 | C-T | Silent | 50% | | rs7135947 |
| 2503 | A-G | M835V | 4% | (1/24) | |
| 6080 | C-T | T2027I | 17% | (14/83) | az0006826 |
| 6108 | C-G | F2036L | 1.5% | (1/56) | |
| 6204 | C-T | silent | 35% | | rs3742023 |
| 6421 | A-G | I2141V | 19% | (16/83) | rs2075260 |

11 polymorphisms were seen in the coding sequence of ACC2 (SEQ ID No. 2). In addition to these polymorphisms there is a db SNP rs2241220, C-T at position 4506. All liver sequences were homozygous C, whereas SEQ. ID No. 2 has a T in that position. Additionally, SEQ. ID No. 2 has the lower frequency allele at positions 525 and 1791. However, these three positions do not affect the amino acid sequence. We have also identified an A-G polymorphism (rs2075262) at the 9^{th} base of the 3′ UTR with about 50% allele frequency, and SNPs in intron 10 (rs7978168), allele frequency 7% and in intron 41 (rs2075259) allele frequency 20%.

TABLE 2
Haplotype analysis for the 3 common amino acid
changing SNPs indicates five haplotypes.

| | Position | | | |
|---|---|---|---|---|
| | 1951 | 6080 | 6421 | Frequency |
| Haplotype 1 | G | C | A | 35/66 |
| Haplotype 2 | A | C | A | 12/66 |
| Haplotype 3 | G | C | G | 12/66 |
| Haplotype 4 | G | T | A | 5/66 |
| Haplotype 5 | A | T | A | 2/66 |

The sequence shown in Seq. ID 2 represents the most common haplotype 1.

14 individuals will give a 95% probability of detecting an allele of 10% frequency. It is unlikely that there are other SNPs affecting the estimation of which sequence that is the most common sequence. Haplotype analysis demonstrates five different protein sequences wherein three sequences are likely to be common within the population. Haplotype 1 is the most common but only represents 50% of chromosomes. The amino acid substitutions are conservative.

1. An isolated nucleic acid molecule comprising a nucleotide sequence that encodes an acetyl CoA carboxylase 2 (ACC2) splice variant having the amino acid sequence according to SEQ ID NO: 1, or a variant having at least 85% sequence identity thereto, or a variant differing from the sequence disclosed in SEQ ID NO: 1, only by the substitution of synonymous codons, which variant lacks membrane binding ability.

2. The isolated nucleic acid according to claim 1, which comprises a nucleotide sequence that encodes the polypeptide comprising SEQ ID NO: 3.

3. The isolated nucleic acid according to claim 2, which comprises a nucleotide sequence that encodes a variant of the polypeptide disclosed in SEQ ID NO: 3 wherein said variant has at least 85% sequence identity thereto.

4. The isolated nucleic acid according to claim 1, which comprises the nucleotide sequence depicted in SEQ ID NO:5.

5. A plasmid comprising the nucleic acid according to claim 1.

6. An isolated cell or cell line comprising the nucleic acid according to claim 1.

7. An isolated cell or cell line transformed or transfected with the nucleic acid according to claim 1.

8. The cell or cell line according to claim 6, which is a mammalian, bacterial, yeast or insect cell or cell line.

9. A method for producing a polypeptide comprising the amino acid sequence of SEQ ID NO: 1, a sequence with at least 85% sequence identity thereto, or C-terminal truncated versions thereof, said polypeptide being incapable of membrane anchoring, the method comprising:
a) culturing a host cell containing an expression vector comprising a nucleic acid sequence which encodes a ACC2 splice variant polypeptide, or a polypeptide with at least 85% sequence identity thereto, or C-terminal truncated version thereof, which polypeptide is incapable of membrane anchoring, under conditions suitable for expression of the polypeptide; and
b) recovering the polypeptide from the host cell culture.

10. An isolated polypeptide comprising the amino acid sequence depicted in SEQ ID NO: 1, or a sequence possessing at least 85% similarity thereto, which polypeptide is incapable of membrane binding.

11. The isolated polypeptide according to claim 10, which comprises the amino acid sequence depicted in SEQ ID NO: 1.

12. An isolated ACC2-ACC2(1b) protein complex.

13. A purified antibody, capable of selectively binding to an ACC2 splice variant.

14. An antibody according to claim 13, capable of selectively binding to the ACC2(1b) splice variant.

15. (canceled)

16. A method of treating a human having a disease or disorder associated with impaired ability to oxidise fatty acids, comprising administering to the human an inhibitory nucleic acid molecule against an ACC2 splice variant or a selective antibody directed against ACC2 splice variant protein.

17. The method as claimed in claim 16, wherein the disease or disorder associated with impaired ability to oxidise fatty acids is selected from the group consisting of: obesity, type 2 diabetes mellitus and dyslipidaemia.

18. A method of identifying a compound potentially useful for treating a disease or disorder associated with impaired ability to oxidise fatty acids, which comprises assaying the compound for its ability to modulate the activity or amount of an ACC2 splice variant protein.

19. The method according to claim 18, wherein the assay is selected from:
i) measurement of ACC2 splice variant activity using a cell line which expresses ACC2 splice variant or using purified ACC2 splice variant protein; and
ii) measurement of ACC2 splice variant transcription or translation in a cell line expressing ACC2 splice variant.

20. (canceled)

21. A method of identifying a compound potentially useful for treating a disease or disorder associated with impaired ability to oxidise fatty acids, which comprises assaying the compound for its ability to modulate the activity or amount of a complex comprising a ACC2 and an ACC2 splice variant.

22. The method according to claim 21, using a isolated ACC2 and an ACC2(1b) splice variant complex and measuring the activity of said complex with respect to malonyl CoA production.

23. The method of claim 22, wherein said method comprises the measurement of malachite green detection of produced inorganic phosphate formed by said complex.

24. The method of claim 22, wherein said method comprises measurement of the incorporation of ¹⁴CO₂ into ¹⁴C-malonyl CoA.

25. The method according to claim 21, comprising: measurement of the activity of the ACC2 and ACC2(1b) splice variant complex using a cell line which expresses the ACC2 and ACC2(1b) splice variant complex.

26. The method according to claim 21, comprising measurement of the transcription and/or translation of the ACC2 and ACC2(1b) splice variant complex using a cell line which expresses the ACC2 and ACC2(1b) splice variant complex.

27. A method according to claim 18, wherein said disease or disorder is obesity, type 2 diabetes mellitus, or dyslipidaemia.

28. (canceled)

29. A method of preparing a pharmaceutical composition, which comprises:
(i) identifying a compound as useful for treating diseases such as obesity, type 2 diabetes mellitus, dyslipidaemia, and other disorders associated with impaired ability to oxidise fatty acids according to the method of claim 18; and
(ii) mixing the compound or a pharmaceutically acceptable salt thereof with a pharmaceutically acceptable excipient or diluent.

30-31. (canceled)

32. A method according to claim 21, wherein said disease or disorder is obesity, type 2 diabetes mellitus, or dyslipidaemia.

33. A method of preparing a pharmaceutical composition, which comprises:
(i) identifying a compound as useful for treating diseases such as obesity, type 2 diabetes mellitus, dyslipidaemia, and other disorders associated with impaired ability to oxidise fatty acids according to the method of claim 21; and
(ii) mixing the compound or a pharmaceutically acceptable salt thereof with a pharmaceutically acceptable excipient or diluent.


SEQUENCE LISTING (16 sequences)

SEQ ID NO: 1 - protein, 2256 aa, Human ACC2b polypeptide
MSPAKCKICFPDREVKPSMSGLHLVKRGREHKKLDLHRDFTVDSPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPQSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGVVCGALNVADAMFRTCMTDFLHSLERGQVLPADSLLNLVDVELIYGGVKYILKVARQSLTMFVLIMNGCHIEIDAHRLNDGGLLLSYNGNSYTTYMKEEVDSYRITIGNKTCVFEKENDPTVLRSPSAGKLTQYTVEDGGHVEAGSSYAEMEVMKMIMTLNVQERGRVKYIKRPGAVLEAGCVVARLELDDPSKVHPAEPFTGELPAQQTLPILGEKLHQVFHSVLENLTNVMSGFCLPEPVFSIKLKEWVQKLMMTLRHPSLPLLELQEIMTSVAGRIPAPVEKSVRRVMAQYASNITSVLCQFPSQQIATILDCHAATLQRKADREVFFINTQSIVQLVQRYRSGIRGYMKTVVLDLLRRYLRVEHHFQQAHYDKCVINLREQFKPDMSQVLDCIFSHAQVAKKNQLVIMLIDELCGPDPSLSDELISILNELTQLSKSEHCKVALRARQILIASHLPSYELRHNQVESIFLSAIDMYGHQFCPENLKKLILSETTIFDVLPTFFYHANKVVCMASLEVYVRRGYIAYELNSLQHRQLPDGTCVVEFQFMLPSSHPNRMTVPISITNPDLLRHSTELFMDSGFSPLCQRMGAMVAFRRFEDFTRNFDEVISCFANVPKDTPLFSEARTSLYSEDDCKSLREEPIHILNVSIQCADHLEDEALVPILRTFVQSKKNILVDYGLRRITFLIAQEKEFPKFFTFRARDEFAEDRIYRHLEPALAFQLELNRMRNFDLTAVPCANHKMHLYLGAAKVKEGVEVTDHRFFIRAIIRHSDLITKEASFEYLQNEGERLLLEAMDELEVAFNNTSVRTDCNHIFLNFVPTVIMDPFKIEESVRYMVMRYGSRLWKLRVLQAEVKINIRQTTTGSAVPIRLFITNESGYYLDISLYKEVTDSRSGNIMFHSFGNKQGPQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST

SEQ ID NO: 2 - DNA, 7377 bp, human ACC2 cDNA
atggtcttgcttctttgtctatcttgtctgattttctcctgtctgaccttttcctggttaaaaatctgggggaaaatgacggactccaagccgatcaccaagagtaaatcagaagcaaacctcatcccgagccaggagccctttccagcctctgataactcaggggagacaccgcagagaaatggggagggccacactctgcccaagacacccagccaggccgagccagcctcccacaaaggccccaaagatgccggtcggcggagaaactccctaccaccctcccaccagaagcccccaagaaaccccctttcttccagtgacgcagcaccctccccagagcttcaagccaacgggactgggacacaaggtctggaggccacagataccaatggcctgtcctcctcagccaggccccagggccagcaagctggctccccctccaaagaagacaagaagcaggcaaacatcaagaggcagctgatgaccaacttcatcctgggctcttttgatgactactcctccgacgaggactctgttgctggctcatctcgtgagtctacccggaagggcagccgggccagcttgggggccctgtccctggaggcttatctgaccacaggtgaagctgagacccgcgtccccactatgaggccgagcatgtcgggactccacctggtgaagaggggacgggaacacaagaagctggacctgcacagagactttaccgtggcttctcccgctgagtttgtcacacgctttgggggggatcgggtcatcgagaaggtgcttattgccaacaacgggattgccgccgtgaagtgcatgcgctccatccgcaggtgggcctatgagatgttccgcaacgagcgggccatccggtttgttgtgatggtgacccccgaggaccttaaggccaacgcagagtacatcaagatggcggatcattacgtccccgtcccaggagggcccaataacaacaactatgccaacgtggagctgattgtggacattgccaagagaatccccgtgcaggcggtgtgggctggctggggccatgcttcagaaaaccctaaacttccggagctgctgtgcaagaatggagttgctttcttaggccctcccagtgaggccatgtgggccttaggagataagatcgcctccaccgttgtcgcccagacgctacaggtcccaaccctgccctggagtggaagcggcctgacagtggagtggacagaagatgatctgcagcagggaaaaagaatcagtgtcccagaagatgtttatgacaagggttgcgtgaaagacgtagatgagggcttggaggcagcagaaagaattggttttccattgatgatcaaagcttctgaaggtggcggagggaagggaatccggaaggctgagagtgcggaggacttcccgatccttttcagacaagtacagagtgagatcccaggctcgcccatctttctcatgaagctggcccagcacgcccgtcacctggaagttcagatcctcgctgaccagtatgggaatgctgtgtctctgtttggtcgcgactgctccatccagcggcggcatcagaagatcgttgaggaagcaccggccaccatcgccccgctggccatattcgagttcatggagcagtgtgccatccgcctggccaagaccgtgggctatgtgagtgcagggacagtggaatacctctatagtcaggatggcagcttccacttcttggagctgaatcctcgcttgcaggtggaacatccctgcacagaaatgattgctgatgttaatctgccggccgcccagctacagatcgccatgggcgtgccactgcaccggctgaaggatatccggcttctgtatggagagtcaccatggggagtgactcccatttcttttgaaaccccctcaaaccctcccctcgcccgaggccacgtcattgccgccagaatcaccagcgaaaacccagacgagggttttaagccgagctccgggactgtccaggaactgaatttccggagcagcaagaacgtgtggggttacttcagcgtggccgctactggaggcctgcacgagtttgcggattcccaatttgggcactgcttctcctggggagagaaccgggaagaggccatttcgaacatggtggtggctttgaaggaactgtccatccgaggcgactttaggactaccgtggaatacctcattaacctcctggagaccgagagcttccagaacaacgacatcgacaccgggtggttggactacctcattgctgagaaagtgcaggcggagaaaccggatatcatgcttggggtggtatgcggggccttgaacgtggccgatgcgatgttcagaacgtgcatgacagatttcttacactccctggaaaggggccaggtcctcccagcggattcactactgaacctcgtagatgtggaattaatttacggaggtgttaagtacattctcaaggtggcccggcagtctctgaccatgttcgttctcatcatgaatggctgccacatcgagattgatgcccaccggctgaatgatggggggctcctgctctcctacaatgggaacagctacaccacctacatgaaggaagaggttgacagttaccgaattaccatcggcaataagacgtgtgtgtttgagaaggagaacgatcctacagtcctgagatccccctcggctgggaagctgacacagtacacagtggaggatgggggccacgttgaggctgggagcagctacgctgagatggaggtgatgaagatgatcatgaccctgaacgttcaggaaagaggccgggtgaagtacatcaagcgtccaggtgccgtgctggaagcaggctgcgtggtggccaggctggagctcgatgacccttctaaagtccacccggctgaaccgttcacaggagaactccctgcccagcagacactgcccatcctcggagagaaactgcaccaggtcttccacagcgtcctggaaaacctcaccaacgtcatgagtggcttttgtctgccagagcccgtttttagcataaagctgaaggagtgggtgcagaagctcatgatgaccctccggcacccgtcactgccgctgctggagctgcaggagatcatgaccagcgtggcaggccgcatccccgcccctgtggagaagtctgtccgcagggtgatggcccagtatgccagcaacatcacctcggtgctgtgccagttccccagccagcagatagccaccatcctggactgccatgcagccaccctgcagcggaaggctgatcgagaggtcttcttcatcaacacccagagcatcgtgcagttggtccagagataccgcagcgggatccgcggctatatgaaaacagtggtgttggatctcctgagaagatacttgcgtgttgagcaccattttcagcaagcccactacgacaagtgtgtgataaacctcagggagcagttcaagccagacatgtcccaggtgctggactgcatcttctcccacgcacaggtggccaagaagaaccagctggtgatcatgttgatcgatgagctgtgtggcccagacccttccctgtcggacgagctgatctccatcctcaacgagctcactcagctgagcaaaagcgagcactgcaaagtggccctcagagcccggcagatcctgattgcctcccacctcccctcctacgagctgcggcataaccaggtggagtccattttcctgtctgccattgacatgtacggccaccagttctgccccgagaacctcaagaaattaatactttcggaaacaaccatcttcgacgtcctgcctactttcttctatcacgcaaacaaagtcgtgtgcatggcgtccttggaggtttacgtgcggaggggctacatcgcctatgagttaaacagcctgcagcaccggcagctcccggacggcacctgcgtggtagaattccagttcatgctgccgtcctcccacccaaaccggatgaccgtgcccatcagcatcaccaaccctgacctgctgaggcacagcacagagctcttcatggacagcggcttctccccactgtgccagcgcatgggagccatggtagccttcaggagattcgaggacttcaccagaaattttgatgaagtcatctcttgcttcgccaacgtgcccaaagacacccccctcttcagcgaggcccgcacctccctatactccgaggatgactgcaagagcctcagagaagagcccatccacattctgaatgtgtccatccagtgtgcagaccacctggaggatgaggcactggtgccgattttacggacattcgtacagtccaagaaaaatatccttgtggattatggactccgacgaatcacattcttgattgcccaagagaaagaatttcccaagtttttcacattcagagcaagagatgagtttgcagaagatcgcatttaccgtcacttggaacctgccctggccttccagctggaacttaaccggatgcgtaacttcgatctgaccgccgtgccctgtgccaaccacaagatgcacctttacctgggtgctgccaaggtgaaggaaggtgtggaagtgacggaccataggttcttcatccgcgccatcatcaggcactctgacctgatcacaaaggaagcctccttcgaatacctgcagaacgagggtgagcggctgctcctggaggccatggacgagctggaggtggcgttcaataacaccagcgtgcgcaccgactgcaaccacatcttcctcaacttcgtgcccactgtcatcatggaccccttcaagatcgaggagtccgtgcgctacatggttatgcgctacggcagccggctgtggaaactccgtgtgctacaggctgaggtcaagatcaacatccgccagaccaccaccggcagtgccgttcccatccgcctgttcatcaccaatgagtcgggctactacctggacatcagcctctacaaagaagtgactgactccagatctggaaatatcatgtttcactccttcggcaacaagcaagggccccagcacgggatgctgatcaatactccctacgtcaccaaggatctgctccaggccaagcgattccaggcccagaccctgggaaccacctacatctatgacttcccggaaatgttcaggcaggctctctttaaactgtggggctccccagacaagtatcccaaagacatcctgacatacactgaattagtgttggactctcagggccagctggtggagatgaaccgacttcctggtggaaatgaggtgggcatggtggccttcaaaatgaggtttaagacccaggagtacccggaaggacgggatgtgatcgtcatcggcaatgacatcacctttcgcattggatcctttggccctggagaggaccttctgtacctgcgggcatccgagatggcccgggcagagggcattcccaaaatttacgtggcagccaacagtggcgcccgtattggcatggcagaggagatcaaacacatgttccacgtggcttgggtggacccagaagacccccacaaaggatttaaatacctgtacctgactccccaagactacaccagaatcagctccctgaactccgtccactgtaaacacatcgaggaaggaggagagtccagatacatgatcacggatatcatcgggaaggatgatggcttgggcgtggagaatctgaggggctcaggcatgattgctggggagtcctctctggcttacgaagagatcgtcaccattagcttggtgacctgccgagccattgggattggggcctacttggtgaggctgggccagcgagtgatccaggtggagaattcccacatcatcctcacaggagcaagtgctctcaacaaggtcctgggaagagaggtctacacatccaacaaccagctgggtggcgttcagatcatgcattacaatggtgtctcccacatcaccgtgccagatgactttgagggggtttataccatcctggagtggctgtcctatatgccaaaggataatcacagccctgtccctatcatcacacccactgaccccattgacagagaaattgaattcctcccatccagagctccctacgacccccggtggatgcttgcaggaaggcctcacccaactctgaagggaacgtggcagagcggattctttgaccacggcagtttcaaggaaatcatggcaccctgggcgcagaccgtggtgacaggacgagcaaggcttggggggattcccgtgggagtgattgctgtggagacacggactgtggaggtggcagtccctgcagaccctgccaacctggattctgaggccaagataattcagcaggcaggacaggtgtggttcccagactcagcctacaaaaccgcccaggccatcaaggacttcaaccgggagaagttgcccctgatgatctttgccaactggagggggttctccggtggcatgaaagacatgtatgaccaggtgctgaagtttggagcctacatcgtggacggccttagacaatacaaacagcccatcctgatctatatcccgccctatgcggagctccggggaggctcctgggtggtcatagatgccaccatcaacccgctgtgcatagaaatgtatgcagacaaagagagcaggggtggtgttctggaaccagaggggacagtggagattaagttccgaaagaaagatctgataaagtccatgagaaggatcgatccagcttacaagaagctcatggaacagctaggggaacctgatctctccgacaaggaccgaaaggacctggagggccggctaaaggctcgcgaggacctgctgctccccatctaccaccaggtggcggtgcagttcgccgacttccatgacacacccggccggatgctggagaagggcgtcatatctgacatcctggagtggaagaccgcacgcaccttcctgtattggcgtctgcgccgcctcctcctggaggaccaggtcaagcaggagatcctgcaggccagcggggagctgagtcacgtgcatatccagtccatgctgcgtcgctggttcgtggagacggagggggctgtcaaggcctacttgtgggacaacaaccaggtggttgtgcagtggctggaacagcactggcaggcaggggatggcccgcgctccaccatccgtgagaacatcacgtacctgaagcacgactctgtcctcaagaccatccgaggcctggttgaagaaaaccccgaggtggccgtggactgtgtgatatacctgagccagcacatcagcccagctgagcgggcgcaggtcgttcacctgctgtctaccatggacagcccggcctccacctga

SEQ ID NO: 3 - protein, 17 aa, human ACC2(1b) N-terminus
MSPAKCKICFPDREVKP

SEQ ID NO: 4 - protein, 8 aa, predicted human ACC2 membrane/mitochondrial targeting sequence
MVLLLCLS

SEQ ID NO: 5 - DNA, 6762 bp, human ACC2(1b) sequence
atgagtcctgccaagtgcaagatctgtttccctgatcgcgaagtaaagtcgggactccacctggtgaagaggggacgggaacacaagaagctggacctgcacagagactttaccgtggcttctcccgctgagtttgtcacacgctttgggggggatcgggtcatcgagaaggtgcttattgccaacaacgggattgccgccgtgaagtgcatgcgctccatccgcaggtgggcctatgagatgttccgcaacgagcgggccatccggtttgttgtgatggtgacccccgaggaccttaaggccaacgcagagtacatcaagatggcggatcattacgtccccgtcccaggagggcccaataacaacaactatgccaacgtggagctgattgtggacattgccaagagaatccccgtgcaggcggtgtgggctggctggggccatgcttcagaaaaccctaaacttccggagctgctgtgcaagaatggagttgctttcttaggccctcccagtgaggccatgtgggccttaggagataagatcgcctccaccgttgtcgcccagacgctacaggtcccaaccctgccctggagtggaagcggcctgacagtggagtggacagaagatgatctgcagcagggaaaaagaatcagtgtcccagaagatgtttatgacaagggttgcgtgaaagacgtagatgagggcttggaggcagcagaaagaattggttttccattgatgatcaaagcttctgaaggtggcggagggaagggaatccggaaggctgagagtgcggaggacttcccgatccttttcagacaagtacagagtgagatcccaggctcgcccatctttctcatgaagctggcccagcacgcccgtcacctggaagttcagatcctcgctgaccagtatgggaatgctgtgtctctgtttggtcgcgactgctccatccagcggcggcatcagaagatcgttgaggaagcaccggccaccatcgccccgctggccatattcgagttcatggagcagtgtgccatccgcctggccaagaccgtgggctatgtgagtgcagggacagtggaatacctctatagtcaggatggcagcttccacttcttggagctgaatcctcgcttgcaggtggaacatccctgcacagaaatgattgctgatgttaatctgccggccgcccagctacagatcgccatgggcgtgccactgcaccggctgaaggatatccggcttctgtatggagagtcaccatggggagtgactcccatttcttttgaaaccccctcaaaccctcccctcgcccgaggccacgtcattgccgccagaatcaccagcgaaaacccagacgagggttttaagccgagctccgggactgtccaggaactgaatttccggagcagcaagaacgtgtggggttacttcagcgtggccgctactggaggcctgcacgagtttgcggattcccaatttgggcactgcttctcctggggagagaaccgggaagaggccatttcgaacatggtggtggctttgaaggaactgtccatccgaggcgactttaggactaccgtggaatacctcattaacctcctggagaccgagagcttccagaacaacgacatcgacaccgggtggttggactacctcattgctgagaaagtgcaggcggagaaaccggatatcatgcttggggtggtatgcggggccttgaacgtggccgatgcgatgttcagaacgtgcatgacagatttcttacactccctggaaaggggccaggtcctcccagcggattcactactgaacctcgtagatgtggaattaatttacggaggtgttaagtacattctcaaggtggcccggcagtctctgaccatgttcgttctcatcatgaatggctgccacatcgagattgatgcccaccggctgaatgatggggggctcctgctctcctacaatgggaacagctacaccacctacatgaaggaagaggttgacagttaccgaattaccatcggcaataagacgtgtgtgtttgagaaggagaacgatcctacagtcctgagatccccctcggctgggaagctgacacagtacacagtggaggatgggggccacgttgaggctgggagcagctacgctgagatggaggtgatgaagatgatcatgaccctgaacgttcaggaaagaggccgggtgaagtacatcaagcgtccaggtgccgtgctggaagcaggctgcgtggtggccaggctggagctcgatgacccttctaaagtccacccggctgaaccgttcacaggagaactccctgcccagcagacactgcccatcctcggagagaaactgcaccaggtcttccacagcgtcctggaaaacctcaccaacgtcatgagtggcttttgtctgccagagcccgtttttagcataaagctgaaggagtgggtgcagaagctcatgatgaccctccggcacccgtcactgccgctgctggagctgcaggagatcatgaccagcgtggcaggccgcatccccgcccctgtggagaagtctgtccgcagggtgatggcccagtatgccagcaacatcacctcggtgctgtgccagttccccagccagcagatagccaccatcctggactgccatgcagccaccctgcagcggaaggctgatcgagaggtcttcttcatcaacacccagagcatcgtgcagttggtccagagataccgcagcgggatccgcggctatatgaaaacagtggtgttggatctcctgagaagatacttgcgtgttgagcaccattttcagcaagcccactacgacaagtgtgtgataaacctcagggagcagttcaagccagacatgtcccaggtgctggactgcatcttctcccacgcacaggtggccaagaagaaccagctggtgatcatgttgatcgatgagctgtgtggcccagacccttccctgtcggacgagctgatctccatcctcaacgagctcactcagctgagcaaaagcgagcactgcaaagtggccctcagagcccggcagatcctgattgcctcccacctcccctcctacgagctgcggcataaccaggtggagtccattttcctgtctgccattgacatgtacggccaccagttctgccccgagaacctcaagaaattaatactttcggaaacaaccatcttcgacgtcctgcctactttcttctatcacgcaaacaaagtcgtgtgcatggcgtccttggaggtttacgtgcggaggggctacatcgcctatgagttaaacagcctgcagcaccggcagctcccggacggcacctgcgtggtagaattccagttcatgctgccgtcctcccacccaaaccggatgaccgtgcccatcagcatcaccaaccctgacctgctgaggcacagcacagagctcttcatggacagcggcttctccccactgtgccagcgcatgggagccatggtagccttcaggagattcgaggacttcaccagaaattttgatgaagtcatctcttgcttcgccaacgtgcccaaagacacccccctcttcagcgaggcccgcacctccctatactccgaggatgactgcaagagcctcagagaagagcccatccacattctgaatgtgtccatccagtgtgcagaccacctggaggatgaggcactggtgccgattttacggacattcgtacagtccaagaaaaatatccttgtggattatggactccgacgaatcacattcttgattgcccaagagaaagaatttcccaagtttttcacattcagagcaagagatgagtttgcagaagatcgcatttaccgtcacttggaacctgccctggccttccagctggaacttaaccggatgcgtaacttcgatctgaccgccgtgccctgtgccaaccacaagatgcacctttacctgggtgctgccaaggtgaaggaaggtgtggaagtgacggaccataggttcttcatccgcgccatcatcaggcactctgacctgatcacaaaggaagcctccttcgaatacctgcagaacgagggtgagcggctgctcctggaggccatggacgagctggaggtggcgttcaataacaccagcgtgcgcaccgactgcaaccacatcttcctcaacttcgtgcccactgtcatcatggaccccttcaagatcgaggagtccgtgcgctacatggttatgcgctacggcagccggctgtggaaactccgtgtgctacaggctgaggtcaagatcaacatccgccagaccaccaccggcagtgccgttcccatccgcctgttcatcaccaatgagtcgggctactacctggacatcagcctctacaaagaagtgactgactccagatctggaaatatcatgtttcactccttcggcaacaagcaagggccccagcacgggatgctgatcaatactccctacgtcaccaaggatctgctccaggccaagcgattccaggcccagaccctgggaaccacctacatctatgacttcccggaaatgttcaggcaggctctctttaaactgtggggctccccagacaagtatcccaaagacatcctgacatacactgaattagtgttggactctcagggccagctggtggagatgaaccgacttcctggtggaaatgaggtgggcatggtggccttcaaaatgaggtttaagacccaggagtacccggaaggacgggatgtgatcgtcatcggcaatgacatcacctttcgcattggatcctttggccctggagaggaccttctgtacctgcgggcatccgagatggcccgggcagagggcattcccaaaatttacgtggcagccaacagtggcgcccgtattggcatggcagaggagatcaaacacatgttccacgtggcttgggtggacccagaagacccccacaaaggatttaaatacctgtacctgactccccaagactacaccagaatcagctccctgaactccgtccactgtaaacacatcgaggaaggaggagagtccagatacatgatcacggatatcatcgggaaggatgatggcttgggcgtggagaatctgaggggctcaggcatgattgctggggagtcctctctggcttacgaagagatcgtcaccattagcttggtgacctgccgagccattgggattggggcctacttggtgaggctgggccagcgagtgatccaggtggagaattcccacatcatcctcacaggagcaagtgctctcaacaaggtcctgggaagagaggtctacacatccaacaaccagctgggtggcgttcagatcatgcattacaatggtgtctcccacatcaccgtgccagatgactttgagggggtttataccatcctggagtggctgtcctatatgccaaaggataatcacagccctgtccctatcatcacacccactgaccccattgacagagaaattgaattcctcccatccagagctccctacgacccccggtggatgcttgcaggaaggcctcacccaactctgaagggaacgtggcagagcggattctttgaccacggcagtttcaaggaaatcatggcaccctgggcgcagaccgtggtgacaggacgagcaaggcttggggggattcccgtgggagtgattgctgtggagacacggactgtggaggtggcagtccctgcagaccctgccaacctggattctgaggccaagataattcagcaggcaggacaggtgtggttcccagactcagcctacaaaaccgcccaggccatcaaggacttcaaccgggagaagttgcccctgatgatctttgccaactggagggggttctccggtggcatgaaagacatgtatgaccaggtgctgaagtttggagcctacatcgtggacggccttagacaatacaaacagcccatcctgatctatatcccgccctatgcggagctccggggaggctcctgggtggtcatagatgccaccatcaacccgctgtgcatagaaatgtatgcagacaaagagagcaggggtggtgttctggaaccagaggggacagtggagattaagttccgaaagaaagatctgataaagtccatgagaaggatcgatccagcttacaagaagctcatggaacagctaggggaacctgatctctccgacaaggaccgaaaggacctggagggccggctaaaggctcgcgaggacctgctgctccccatctaccaccaggtggcggtgcagttcgccgacttccatgacacacccggccggatgctggagaagggcgtcatatctgacatcctggagtggaagaccgcacgcaccttcctgtattggcgtctgcgccgcctcctcctggaggaccaggtcaagcaggagatcctgcaggccagcggggagctgagtcacgtgcatatccagtccatgctgcgtcgctggttcgtggagacggagggggctgtcaaggcctacttgtgggacaacaaccaggtggttgtgcagtggctggaacagcactggcaggcaggggatggcccgcgctccaccatccgtgagaacatcacgtacctgaagcacgactctgtcctcaagaccatccgaggcctggttgaagaaaaccccgaggtggccgtggactgtgtgatatacctgagccagcacatcagcccagctgagcgggcgcaggtcgttcacctgctgtctaccatggacagcccggcctccacctga

SEQ ID NO: 6 - DNA, 23 bp, forward PCR primer for human ACC2
atggtcttgcttctttgtctatc

SEQ ID NO: 7 - DNA, 21 bp, reverse PCR primer for human ACC2
ggctgtttaacacataggcga

SEQ ID NO: 8 - DNA, 20 bp, forward PCR primer for human ACC2
cagagcatggtgcagttggt

SEQ ID NO: 9 - DNA, 22 bp, reverse pcr primer for human acc2
ccatgtcttccaggagaggtcc

SEQ ID NO: 10 - DNA, 20 bp, forward PCR primer for human ACC2
tcgtcatcggcaatgacata

SEQ ID NO: 11 - DNA, 16 bp, reverse PCR primer for human ACC2
ggtccacctccggccc

SEQ ID NO: 12 - DNA, 22 bp, forward PCR primer for human ACC2(1b)
agtcctgccaagtgcaagatct

SEQ ID NO: 13 - skipped

SEQ ID NO: 14 - DNA, 26 bp, probe for human ACC2(1b)
tgatcgcgaagtaaagccgagcatgt

SEQ ID NO: 15 - DNA, 35 bp, forward PCR primer for human ACC2/ACC2(1b) sequence
ataagctagcgccaccatgagtcctgccaagtgca

SEQ ID NO: 16 - DNA, 27 bp, reverse pcr primer for human acc2/acc2(1b) sequence
tcctccgcactctcagccttccggatt